(19) United States
(12) Patent Application Publication
(10) Pub. No.: US 2012/0116177 A1
(43) Pub. Date: May 10, 2012

(54) APPARATUS AND METHOD FOR CALCULATING CALORIE CONSUMPTION USING 3-AXIAL ACCELEROMETER

(76) Inventors: Hyung-Suk LHO, Suwon-si (KR); We-duk Cho, Seongnam-si (KR); Yang-weon Kim, Incheon (KR); Ik-hyeon Heo, Seoul (KR)

(21) Appl. No.: 13/290,321

(22) Filed: Nov. 7, 2011

(30) Foreign Application Priority Data
Nov. 8, 2010 (KR) 10-2010-0110656
Oct. 24, 2011 (KR) 10-2011-0108560

(51) Int. Cl. A61B 5/00

(52) U.S. Cl. 600/300

(57) ABSTRACT

Disclosed are an apparatus and method for calculating calorie consumption using a 3-dimensional accelerometer, and more particularly, an apparatus and method for calculating calorie consumption using a 3-axial accelerometer, in which energy consumption (Kcal) corresponding to a human's physical activity can be calculated at a high degree of precision.

### CROSS-REFERENCE TO RELATED APPLICATION

This application claims priority to and the benefit of Korean Patent Applications No. 10-2010-0110656 filed on Nov. 8, 2010 and No. 2011-0108560 filed on Oct. 24, 2011 in the Korean Intellectual Property Office, the entire contents of which are incorporated herein by reference.

### BACKGROUND OF THE INVENTION

(a) Field of the Invention

The present invention relates to an apparatus and method for calculating calorie consumption using a 3-dimensional accelerometer, and more particularly, an apparatus and method for calculating calorie consumption using a 3-axial accelerometer, in which energy consumption (Kcal) corresponding to a human's physical activity can be calculated at a high degree of precision.

(b) Description of the Related Art

A human's physical activity is an important factor to keep his/her body in good condition. In other words, the physical activity is important to prevent and cure overweight or obesity, so that energy consumption needed for losing weight and maintaining weight can be achieved through the physical activity.

To lose the weight and maintain the weight, there have been proposed techniques of predicting how much energy (Kcal) the physical activity consumes. Among them, there is a technique of calculating the energy consumption on the basis of acceleration data varied depending on the physical activity.

The calculated energy consumption is combined with user information and used as information for calculating various activity patterns such as activity and a lifestyle. Further, the energy consumption has been much used in measuring health such as measuring exercise or calculating body mass index (BMI) and thus increased in importance.

Accordingly, there has been required a technique for precisely converting the acceleration data varied depending on the physical activity into the energy consumption, and various researches have been continued with regard to a method of calculating the energy consumption through the current 3-axial accelerometer. However, there is much difference between the calculated energy consumption and a user's real energy consumption, and it is recognized as a problem in light of the degree of precision.

### SUMMARY OF THE INVENTION

Accordingly, the present invention is conceived to solve the forgoing problems, and an aspect of the present invention is to provide an apparatus and method for calculating calorie consumption using a 3-axial accelerometer, in which an output value of the 3-axial accelerometer and a predetermined mathematical expression are used to calculate a user's calorie consumption, a lattice wave digital filter is further used to calculate energy without being affected by the acceleration of gravity, and the energy consumption (Kcal) corresponding to a user's physical activity can be calculated at a high degree of precision by taking the above energy and a user's weight and gender into account.

An exemplary embodiment of the present invention provides an apparatus for calculating calorie consumption using a 3-axial accelerometer, the apparatus including: the 3-axial accelerometer which outputs acceleration signals corresponding to a user's physical activity with respect to x-, y- and z-axes; a low band-pass filter which applies low band-pass filtering to the output signals of the accelerometer with respect to each axis; a high band-pass digital filter which applies high band-pass digital filtering to the output signals of the accelerometer filtered by the low band-pass filter; and a calorie consumption calculator which calculates a user's calorie consumption on the basis of the output signal of the accelerometer filtered by the high band-pass digital filter.

The low band-pass filter may include a 2^{nd} order analog low pass filter which applies 2^{nd} order analog filtering to the output signal of the accelerometer with respect to each axis, and a digital low pass filter which applies digital filtering to the output signal of the accelerometer with respect to each axis filtered by the 2^{nd} order analog low pass filter.

The high band-pass digital filter may include a lattice wave digital filter (LWDF), and the lattice wave digital filter includes a 5 Hz high-pass digital filter.

The calorie consumption calculator may include an energy calculator which calculates the sum of energy for a preset time on the basis of the output signal of the accelerometer filtered by the high band-pass digital filter, and an energy consumption calculator which calculates energy consumption corresponding to a user's physical activity on the basis of the sum of energy calculated by the energy calculator and a user's weight and gender.

The energy calculator calculates the sum of energy by the following expression:

Eᵢ=\sqrt{αₓ_{ᵢ}²+α_{y}_{ᵢ}²+α_{z}_{ᵢ}²} [Expression]

where, Ei is energy calculated from the respectively converted signals of the 3-axial accelerometer, and aₓ_{ᵢ}², a_{y}_{ᵢ}², a_{z}_{ᵢ}² are values obtained by raising the signals of the 3-axial accelerometer, respectively converted from the accelerometer signals of the x-, y- and z-axes by the lattice wave digital filters, to the power.

The energy consumption calculator may calculate the energy consumption corresponding to a user's physical activity by the following expression:

Y=W(A+BG)X^{(C-DG)} [Expression]

where, Y is energy consumption corresponding to a user's physical activity; A, B, C and D are real numbers; X is the sum of energy calculated from the signals of the 3-axial accelerometer passed through the lattice wave digital filters corresponding to a user's physical activity for the preset time; and W is a user's weight (kg), and G is a gender index having a value of 0 or 1.

A may be 4.488, B may be 1.869, C may be 0.722, and D may be 0.095.

G may be 0 if a user is a male and 1 if a user is a female.

Another exemplary embodiment of the present invention provides a method for calculating calorie consumption using a 3-axial accelerometer, the method including: receiving output signals of the 3-axial accelerometer corresponding to a user's physical activity and outputting 3-axial accelerometer signals converted by a lattice wave digital filter (LWDF); calculating the sum of energy for a preset time on the basis of the converted 3-axial accelerometer signals; and calculating energy consumption corresponding to a user's physical activity on the basis of the calculated sum of energy and a user's weight and gender.

The lattice wave digital filter may include a 5 Hz high-pass digital filter.

The calculating the sum of energy for a preset time on the basis of the converted 3-axial accelerometer signals may include calculating the sum of energy by the following expression:

Eᵢ=\sqrt{αₓ_{ᵢ}²+α_{y}_{ᵢ}²+α_{z}_{ᵢ}²} [Expression]

where, Ei is energy calculated from the respectively converted signals of the 3-axial accelerometer, and aₓ_{ᵢ}², a_{y}_{ᵢ}², a_{z}_{ᵢ}² are values obtained by raising the signals of the 3-axial accelerometer, respectively converted from the accelerometer signals of the x-, y- and z-axes by the lattice wave digital filters, to the power.

The calculating energy consumption corresponding to a user's physical activity on the basis of the calculated sum of energy and a user's weight and gender may include calculating the energy consumption corresponding to a user's physical activity by the following expression:

Y=W(A+BG)X^{(C-DG)} [Expression]

where, Y is energy consumption corresponding to a user's physical activity; A, B, C and D are real numbers; X is the sum of energy calculated from the signals of the 3-axial accelerometer passed through the lattice wave digital filters corresponding to a user's physical activity for the preset time; and W is a user's weight (kg), and G is a gender index having a value of 0 or 1.

A may be 4.488, B may be 1.869, C may be 0.722, and D may be 0.095.

G may be 0 if a user is a male and 1 if a user is a female.

The method may further include performing zero-adjustment for the 3-axial accelerometer before outputting the converted 3-axial accelerometer signals from the lattice wave digital filter (LWDF) that receives and converts the output signals of the 3-axial accelerometer corresponding to a user's physical activity.

### BRIEF DESCRIPTION OF THE DRAWINGS

The above and/or other aspects of the present invention will become apparent and more readily appreciated from the following description of the exemplary embodiments, taken in conjunction with the accompanying drawings, in which:

FIG. 1 is a block diagram of an apparatus for calculating calorie using a 3-axial accelerometer according to an exemplary embodiment;

FIG. 2 is a waveform showing a result from applying a lattice wave digital filter (LWDF) to the apparatus for calculating calorie using the 3-axial accelerometer according to an exemplary embodiment;

FIG. 3 is a flowchart of a method for calculating calorie using the 3-axial accelerometer according to an exemplary embodiment;

FIG. 4 is a scatter diagram showing calorie (cal) based on gender and the sum (S) of calculated energy according to an exemplary embodiment;

FIG. 5 is a scatter diagram showing calorie divided by weight based on gender (cal/kg) and the sum (S) of calculated energy according to an exemplary embodiment;

FIG. 6 is a graph showing a regression nonlinear relationship of a real measured value of a male user;

FIG. 7 is a graph showing a regression nonlinear relationship of a real measured value of a female user;

FIG. 8 is a table showing characteristics of a subject to acquire experimental data according to an exemplary embodiment;

FIG. 9 is a table showing a test protocol for acquiring experimental data according to an exemplary embodiment;

FIG. 10 is a scatter diagram of Kcal and S according to another exemplary embodiment;

FIG. 11 is a scatter diagram of Kcal/kg and S according to another exemplary embodiment;

FIG. 12 is a scatter diagram of Kcal/Kg and ln(s) according to gender;

FIG. 13 is a table showing a hypothesis testing result according to another exemplary embodiment;

FIG. 14 is a graph showing a residual analysis according to another exemplary embodiment;

FIG. 15 is a graph showing a regression linear relationship between a regression equation and a real measured value;

FIG. 16 is a graph showing a 95% confidence interval for a predicted value of Kcal/Kg; and

FIG. 17 is a table showing a root mean square error (RMSE) and precision with regard to experimental results.

### DETAILED DESCRIPTION OF THE EMBODIMENTS

Hereinafter, an apparatus and method for calculating calorie consumption using a 3-axial accelerometer according to exemplary embodiments will be described with reference to accompanying drawings.

Also, detailed descriptions of known functions and configurations incorporated herein will be omitted if they may make the subject matter of the present invention rather unclear.

Further, since terms to be described are defined in consideration of the functions of the present invention, they may vary according to user's intention, practice or the like. Hence, the terms have to be interpreted based on the contents of the entire specification.

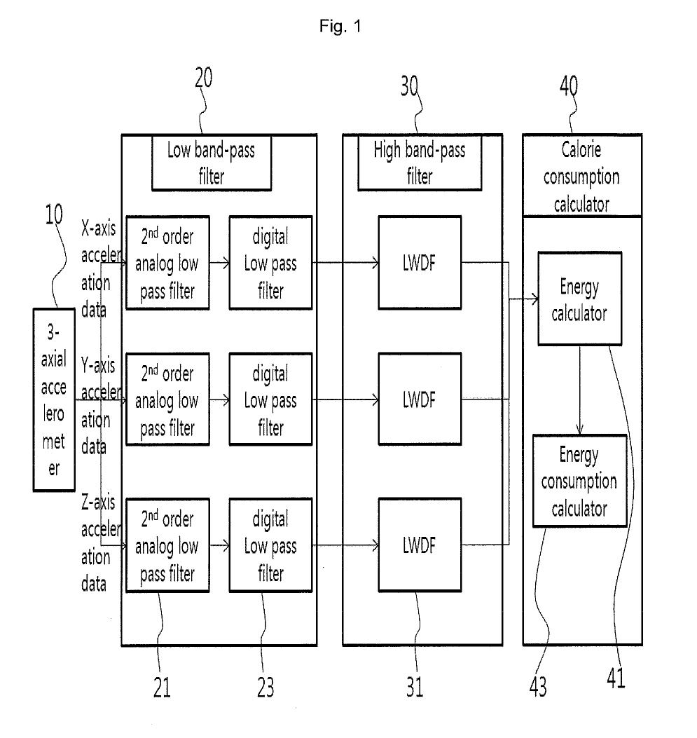

FIG. 1 is a block diagram of an apparatus for calculating calorie using a 3-axial accelerometer according to an exemplary embodiment.

As shown therein, the apparatus for calculating calorie consumption using the 3-axial accelerometer according to this exemplary embodiment includes a 3-axial accelerometer 10, a low band-pass filter 20, a high band-pass filter 30, and a calorie consumption calculator 40.

The 3-axial accelerometer 10 outputs accelerator signals based on a user's physical activity with regard to x-, y- and z-axes if a user performs the physical activity such as motion or exercise. For example, the 3-axial accelerometer 10 outputs X-axis acceleration data, Y-axis acceleration data and Z-axis acceleration data.

The output signals of the accelerometer with regard to each axis, output from the 3-axial accelerometer 10, are input to the low band-pass filter 20. The low band-pass filter 20 applies low band-pass filtering to the signals output from the accelerometer of each axis and outputs them.

As above, if the output signal from the accelerometer of each axis, output from the 3-axial accelerometer 10, is filtered through the low band-pass filter 20, high frequency components such as noise are removed so that calorie consumption can be more precisely calculated.

The low band-pass filter 20, as shown in FIG. 1, performs two-step filtering. That is, the low band-pass filter 20 includes a 2nd order analog low pass filter 21 that applies 2nd order analog filtering to the output signal from the accelerometer of each axis, and a digital low pass filter 23 that applies digital filtering to the output signal from the accelerometer of each axis after being filtered through the 2nd order analog low pass filter 21.

The 2nd order analog low pass filter 21 has a cut-off frequency of 1500 Hz, and thus passes only a frequency lower than 1500 Hz without passing a frequency equal to or higher than 1500 Hz.

Such an analog type 2nd order low pass filter 21 is achieved by hardware, i.e., combination of a resistor, an inductor and a capacitor (RLC). In general, the high frequency components in the signal are not a desired signal but noise components. Thus, the 2nd order analog low pass filter is provided to remove such noise components.

The digital low pass filter 23 may have a cut-off frequency selected among 1500 Hz, 750 Hz, 375 Hz, 190 Hz, 100 Hz, 50 Hz and 25 Hz. Such a digital type low pass filter is achieved by software, i.e., programmed in an accelerometer sensor integrated chip (IC).

A human's motion generally has a frequency of 5˜20 Hz, and thus the cut-off frequency of the digital low pass filter 23 may be set to 25 Hz. However, real time clock (RTC) allows a micro controller unit (MCU) used in this exemplary embodiment to have a timer setup of in the form of squares of 2 (e.g., 2, 4, 8, 16, 32, . . . , 32768), so that 50 Hz higher than 32 can be reasonably selected as the cut-off frequency.

The digital low pass filter 23 also regards frequencies higher than 50 Hz as noise and filters off it.

As above, the output signals of the accelerometer, of which the high frequency components are filtered off by the low band-pass filter 20, are respectively input to the high band-pass digital filter 30. Then, the high band-pass digital filter 30 applies the high band-pass digital filtering to the output signals of the accelerometer which are filtered by the low band-pass filter 20.

If the output signals of the accelerometer which are filtered by the low band-pass filter 20 are filtered again by the high band-pass digital filter 30, the output signals of the accelerometer falling under the acceleration of gravity are removed. In other words, the calorie consumption can be precisely calculated without being affected by the acceleration of gravity.

The high band-pass filter 30 includes three filters to perform the high band-pass digital filtering with regard to the acceleration output signals of the x-, y- and z-axes as shown in FIG. 1.

The high band-pass digital filter 30 includes three lattice wave digital filters (LWDF) 31, and each LWDF is a 5 Hz high-pass digital filter.

The digital filter is broadly classified into a finite impulse response (FIR) filter and an infinite impulse response (IIR) filter. The FIR filter is advantageous since it is stable and has no phase shift. However, the FIR filter has a problem that filter order becomes higher and the amount of calculation increases.

Accordingly, in this exemplary embodiment, the IIR filter is used and the LWDF is used among the IIR filters. While the filtering is performed through the LWDF filter, multiplication, division and fixed-point (floating-point) operations need more load than addition, subtraction and shift operations.

Therefore, the present exemplary embodiment employs the LWDF based on Horner's method and a canonical signal digit (CSD) format. The Horner's method is a method for efficiently processing decimal point multiplication and division. The CSD format is a format for reducing the amount of operation by changing binary numerals so that many additions can be replaced by one addition and one subtraction (refer to [Efficient Multiplication and Division Using MSP430”, TEXAS INSTRUMENTS, SLAA329-September 2006].

The LWDF filter is a kind of the IIR filter, in which four types of adapters are combined to achieve the respective filters (e.g., a low pass filter, a high pass filter, a band-pass filter, etc.). The adapters correspond to only the addition, the subtraction and the multiplication (the multiplication is replaced by the addition and shift operation).

The LWDF based on the Horner's method and the CSD format employed in this exemplary embodiment may be designed by a tool provided by TEXAS INSTRUMENTS.

The output signal of the accelerometer, filtered through the LWDF 31, is input to the calorie consumption calculator 40. Then, the calorie consumption calculator 40 precisely calculates a user's calorie consumption on the basis of the output signal of the accelerometer filtered by the high band-pass digital filter 30 including the LWDFs 31.

As shown in FIG. 1, the calorie consumption calculator 40 includes an energy calculator 41 for calculating the sum of energy for preset time on the basis of the output signal of the accelerometer filtered by the high band-pass digital filter 30, and an energy consumption calculator 43 for calculating energy consumption corresponding to a user's physical activity on the basis of the sum of energy calculated by the energy calculator 41 and a user's weight and gender.

The energy calculator 41 calculates the sum of energy for the preset time on the basis of values output from three LWDFs 31 that respectively perform the filtering with the output signals of the accelerometer with respect to the x-, y- and z-axes.

The energy calculator 41 calculates the sum of energy using the following expression 1.

Eᵢ=\sqrt{αₓ_{ᵢ}²+α_{y}_{ᵢ}²+α_{z}_{ᵢ}²} [Expression 1]

where, Ei is energy calculated from the respectively converted signals of the 3-axial accelerometer, and aₓ_{ᵢ}², a_{y}_{ᵢ}², a_{z}_{ᵢ}² are values obtained by raising the signals of the 3-axial accelerometer, respectively converted from the accelerometer signals of the x-, y- and z-axes by the LWDFs, to the power.

The energy calculated by the expression 1 is an energy value at a certain time, and such a calculation method is repeated for a preset time. Thus, the energy calculator 41 calculates the sum of energy for the preset time on the basis of the expression 1.

As above, the sum of energy calculated by the energy calculator 41 is input to the energy consumption calculator 43. Then, the energy consumption calculator 43 precisely calculates energy consumption corresponding to a user's physical activity on the basis of the sum of energy calculated by the energy calculator 41 and a user's weight and gender.

The energy consumption calculator 43 calculates the energy consumption corresponding to a user's physical activity on the basis of the following expression 2.

Y=W(A+BG)X^{(C-DG)} [Expression 2]

where, Y is energy consumption corresponding to a user's physical activity; A, B, C and D are real numbers; X is the sum of energy calculated from the signals of the 3-axial accelerometer passed through the LWDFs corresponding to a user's physical activity for the preset time; and W is a user's weight (kg), and G is a gender index having a value of 0 or 1.

Here, A may be 4.488, B may be 1.869, C may be 0.722, and D may be 0.095. Further, G may be 0 if a user is a male and 1 if a user is a female.

Using the filters and expressions described above, a problem of error due to the high frequency regarded as noise and the acceleration of gravity can be solved, thereby precisely calculating a user's calorie consumption.

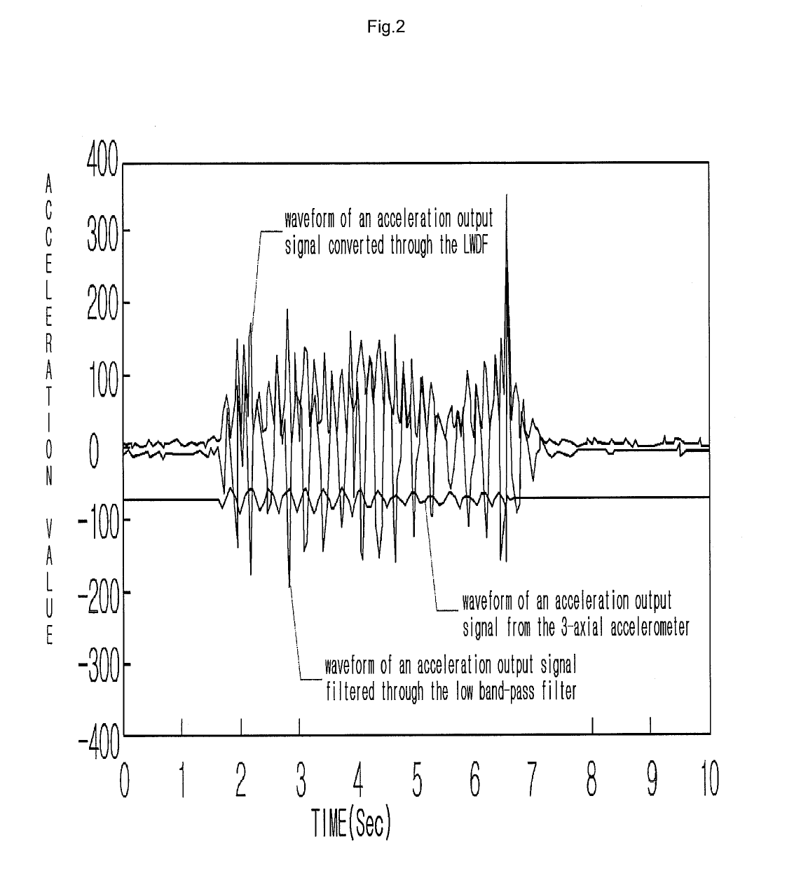

FIG. 2 is a waveform for explaining an effect when the LWDF is used in the apparatus for calculating calorie using the 3-axial accelerometer according to an exemplary embodiment.

Referring to FIG. 2, a blue waveform shows a waveform of an acceleration output signal from the 3-axial accelerometer, a green waveform shows a waveform of an acceleration output signal filtered through the low band-pass filter, and a red waveform is a waveform of an acceleration output signal converted through the LWDF.

As shown therein, the red waveform corresponding to the acceleration output signal filtered through the LWDF shows that a component corresponding to the acceleration of gravity is removed and thus data is offset into 0.

Next, a method of calculating a user's calorie consumption by the apparatus for calculating the calorie consumption using the foregoing 3-axial accelerometer will be described in detail.

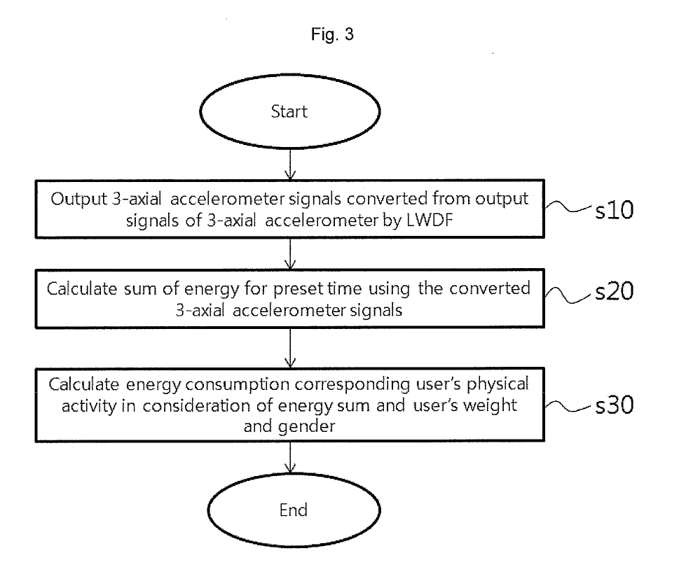

FIG. 3 is a flowchart of a method for calculating calorie using the 3-axial accelerometer according to an exemplary embodiment.

The method for calculating calorie using the 3-axial accelerometer according to an exemplary embodiment employs the apparatus mounted with the 3-axial accelerometer for calculating the calorie consumption. In this calculation method, 3-axial acceleration values output from the 3-axial accelerometer undergo the LWDFs, and the sum of data passed through the LWDF is obtained for the preset time, thereby acquiring the energy consumption corresponding to a user's physical activity on the basis of the sum of energy and a user's weight and gender.

Specifically, as shown in FIG. 3, the output signals of the 3-axial accelerometer corresponding to a user's physical activity are input to and converted by the LWDFs, thereby outputting the 3-axial accelerometer signals (S10). Here, each LWDF is a 5 Hz high-pass digital filter.

That is, the 3-axial accelerometer values are obtained from the output values of the 3-axial accelerometer corresponding to a user's activity by the LWDF. At this time, the LWDF is designed as the 5 Hz high-pass filter, and achieved by an assembler in consideration of the performance of the apparatus mounted with the 3-axial accelerometer for calculating the calorie consumption. Here, the Horner's method using the CSD format is employed in achieving the LWDF.

Meanwhile, the output signal of the 3-axial accelerometer, to be input to the LWDF, is not a raw signal output by the 3-axial accelerometer but the output signal of the 3-axial accelerometer primarily filtered by the low band-pass filter 20 as described with reference to FIG. 1. That is, the output signal of the 3-axial accelerometer, filtered by the low band-pass filter 20, is input to the LWDF.

As above, if the output signal of the 3-axial accelerometer is converted by the LWDF, the energy calculator 41 calculates the sum of energy for the preset time on the basis of the converted 3-axial accelerometer signals (S20). At this time, the energy calculator 41 may use a predetermined mathematical expression to convert the output values of the 3-axial accelerometer (i.e., the output values of the 3-axial accelerometer passed through the LWDF) corresponding to a user's physical activity into energy.

That is, the step of calculating the sum of energy for the preset time using the converted 3-axial accelerometer signals includes calculating the sum of energy using the following expression 1.

Eᵢ=\sqrt{αₓ_{ᵢ}²+α_{y}_{ᵢ}²+α_{z}_{ᵢ}²} [Expression 1]

where, Ei is energy calculated from the respectively converted signals of the 3-axial accelerometer, and aₓ_{ᵢ}², a_{y}_{ᵢ}², a_{z}_{ᵢ}² are values obtained by raising the signals of the 3-axial accelerometer, respectively converted from the accelerometer signals of the x-, y- and z-axes by the LWDFs, to the power. Further, i indicates i^{th} data.

The energy calculator 41 repeats the calculation based on the expression 1 for the preset time, and calculates the sum of total energy for the preset time.

Thus, the sum of energy is input to the energy consumption calculator 43. Then, the energy consumption calculator 43 precisely calculates energy consumption corresponding to a user's physical activity on the basis of the sum of energy and a user's weight and gender (S30).

That is, the energy consumption calculator 43 calculates the energy consumption corresponding to a user's physical activity through the following expression 2 on the basis of the sum of energy and a user's weight and gender.

Y=W(A+BG)X^{(C-DG)} [Expression 2]

where, Y is energy consumption corresponding to a user's physical activity; A, B, C and D are real numbers; X is the sum of energy calculated from the signals of the 3-axial accelerometer passed through the LWDFs corresponding to a user's physical activity for the preset time; and W is a user's weight (kg), and G is a gender index having a value of 0 or 1.

Here, A may be 4.488, B may be 1.869, C may be 0.722, and D may be 0.095. Further, G may be 0 if a user is a male and 1 if a user is a female.

Using the filters and expressions described above, a problem of error due to the high frequency regarded as noise and the acceleration of gravity can be solved, thereby precisely calculating a user's calorie consumption.

Meanwhile, the method of calculating the calorie consumption using the 3-axial accelerometer according to an exemplary embodiment may further include zero adjustment for the 3-axial accelerometer before the step S10, i.e., before the step where the LWDF receives the output signals from the 3-axial accelerometer corresponding to a user's physical activity and outputs the converted 3-axial accelerometer signals.

Below, the energy consumption converting performance of the apparatus for calculating the calorie consumption using the 3-axial accelerometer corresponding to an activity measuring device to which the method for calculating the calorie consumption using the 3-axial accelerometer according to an exemplary embodiment is applied, and the calculation process of the expression 2 for calculating the energy consumption to be used in the apparatus for calculating the calorie consumption using the 3-axial accelerometer will be described.

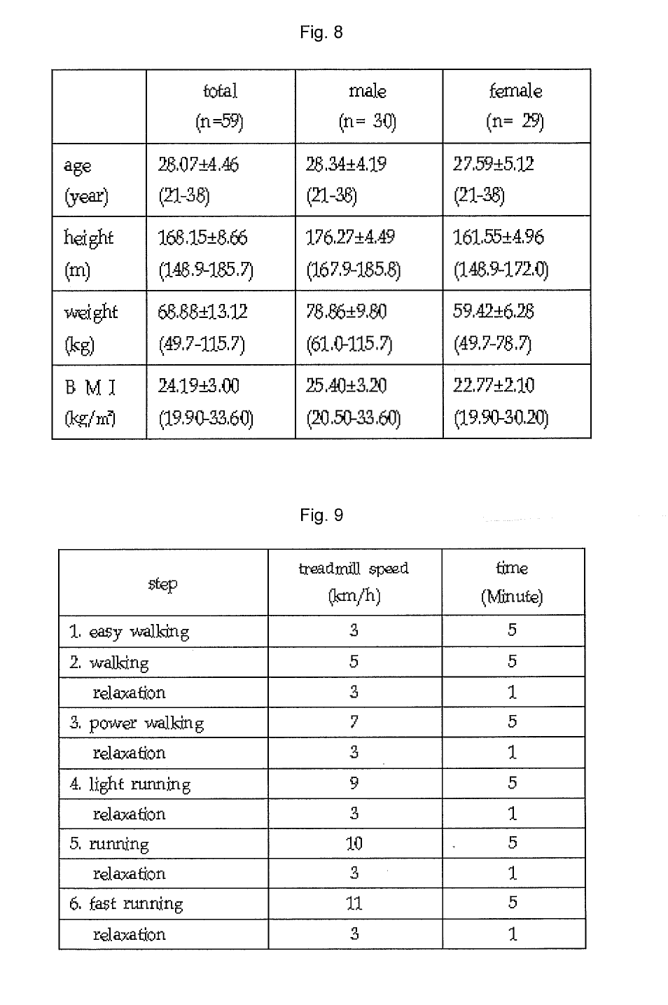

To obtain experimental data, healthy adjusts were recruited as participants in the experiments, and thus 59 men and women between the ages of 21 and 38 were selected. Such subjects have the weights of 49.70 kg to 115.70 kg and an average age of 28. The characteristics of the subjects participated in the present experiments are as shown in FIG. 8, and the acceleration output data of various walking speeds in a treadmill was obtained and tested.

The subjects wore a metabolic gas analyzer (K4B2) and attached an activity monitor to his/her right waist, in which the activity monitor corresponds to the apparatus for calculating the calorie consumption using the 3-axial accelerometer according to this exemplary embodiment. Further, another activity monitor on the market, e.g., the Actical, also were attached to the right waist as a comparable object. Then, the subjects changed speed in order of easy walking, power walking, light running, running and fast running on the treadmill for 5 minutes per step.

A test protocol was acquired through consultation with an exercise physiologist, and interval training of 1 minute was given between the steps in consideration of time to be taken until breathing becomes steady, which were as shown in the following table 2. Taking a physical attribute into account, the treadmill speed of the female was set to be slower by 1 km/h than that of the male.

There is little difference in data output whether the accelerometer is attached to the left or right waist (refer to “N. Twomey, S. Faul, W. P. marnane, Comparison of accelerometer-based energy expenditure estimation algorithms, Pervasive Computing Technologies for Healthcare 4th international conference on, pp 1-8, 2010”). In this exemplary embodiment, the accelerometer was attached to the right waist.

Below, a process of deriving a formula for obtaining a user's energy consumption by using energy converted by the LWDF from the output values of the 3-axial accelerometer corresponding to a user's physical activity based on the test protocol as shown in the table of FIG. 9 will be described.

The 3-axial accelerometer was zero-adjusted using a simple 0g x, 0g y, +1g z calibration method. Then, the LWDF was used for eliminating a component related to the acceleration of gravity since the output values of the 3-axial accelerometer contain the component related to the acceleration of gravity.

Further, because the output values of the 3-axial accelerometer contain a rotation component, the output values was converted into the energy through the foregoing expression 1 so that the rotation component can be processed as one representative value without being considered.

To be matched with the data acquired by the metabolic gas analyzer (K4B2) and the Actical, accelerometer raw data underwent the LWDF in the apparatus for calculating the calorie consumption using the 3-axial accelerometer according to the present exemplary embodiment and was then processed like the following expression 3. Here, n is 1920 as data for one minute, and S is the sum of energy.

$$S = {\sum }_{i = 1}^{n} {E}_{i} \qquad [ Expression 3 ]$$

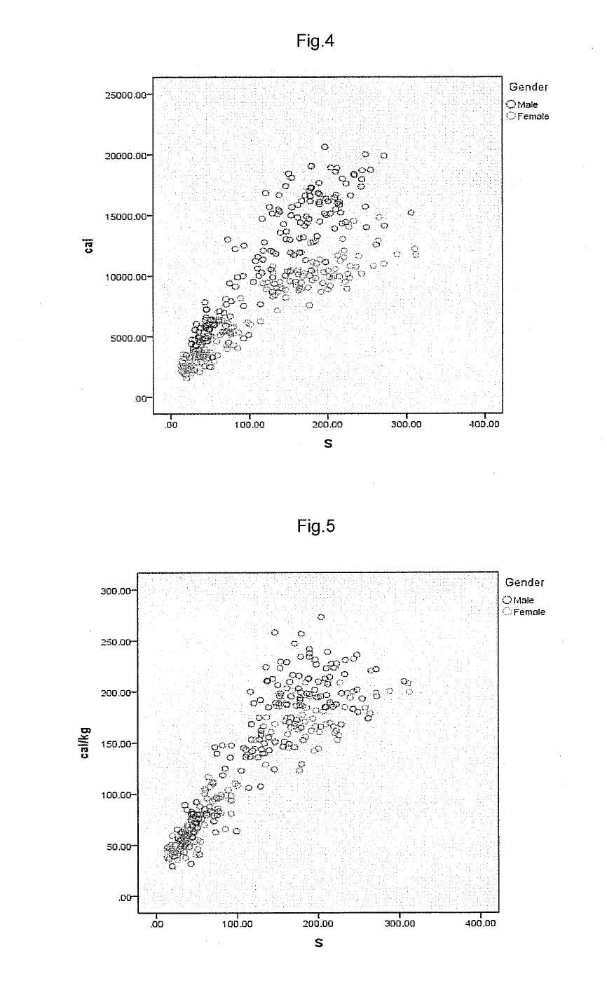

To derive a regression formula, a scatter diagram was drawn using the data acquired through the experiment. FIG. 4 is a scatter diagram showing calorie (cal) based on gender and the sum (S) obtained by the expression 3. It will be appreciated that the male shows higher calorie (cal) than the female with respect to the same S.

Considering that the calorie (cal) is largely dependent on the weight, it is natural since the female has smaller weight than the male. Thus, if a scatter diagram is drawn with S and the calorie (cal) divided by the subject's weight, the scatter diagram shows uniform distribution regardless of gender as shown in FIG. 5. However, it will be appreciated that there is difference in distribution between the male and the female, and the gender is also a variable needed for deriving the regression formula.

Therefore, each curve was estimated according to gender, and it was thus understood that a power model speaks for a relationship between cal/kg and S with regard to both the male and the female. The following table briefly shows the estimated model. Hence, P values (significance probability) of both the male and the female are smaller than 0.05 and thus significant.

| | | | | | Significance |
|---|---|---|---|---|---|
| Gender | R2 | F | df1 | df2 | probability (P) |
| Male | 0.863 | 1061.521 | 1 | 169 | <0.001 |
| Female | 0.907 | 1593.724 | 1 | 164 | <0.001 |

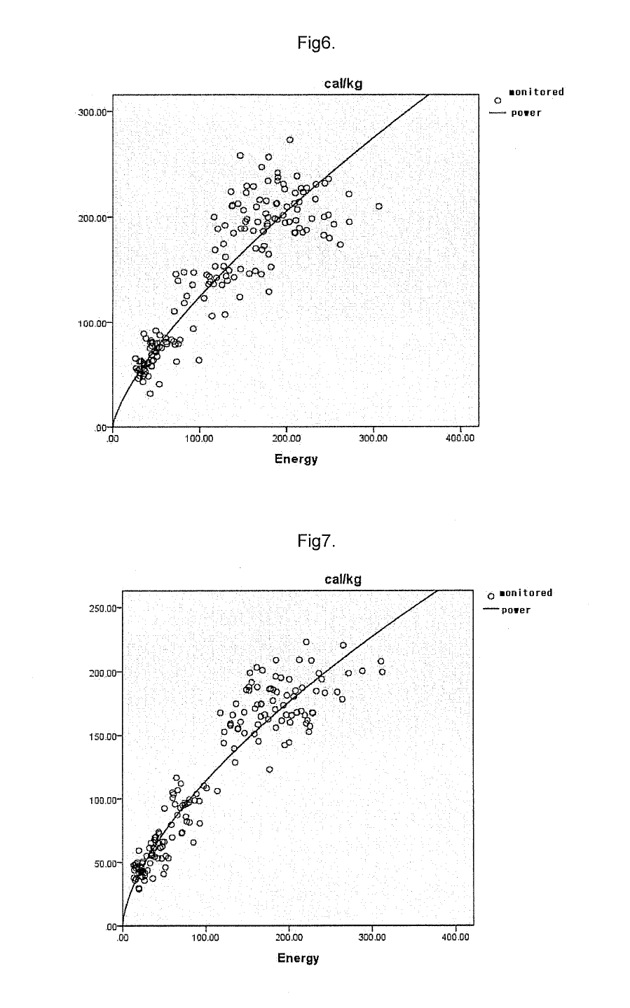

FIG. 6 is a graph showing a regression nonlinear relationship of a real measured value of a male user, and FIG. 7 is a graph showing a regression nonlinear relationship of a real measured value of a female user.

Mean square errors (MSE) in the performances of the expression 2 used in the method of calculating the calorie consumption according to the present exemplary embodiment, AEE1 and AEE2 of the Actical were obtained as shown in the following expression 4, and the precision to the real calorie (cal) from the metabolic gas analyzer was obtained as shown in the following expression 5, which were also tabulated in the following table 4.

$$\begin{matrix}MSE = \frac{1}{n} {\sum }_{i = 1}^{n} {( {Y}_{i} - {\hat{Y}}_{i} )}^{2} & [ Expression 4 ] \\ P = \frac{1}{n} {\sum }_{i = 1}^{n} ( {Y}_{i} - \frac{\vert {Y}_{i} - {\hat{Y}}_{i} \vert}{{Y}_{i}} ) \times 100 & [ Expression 5 ]\end{matrix}$$

n: The number of measured values

Yᵢ: Real calories

Ŷᵢ: Predicted calories

| Sort | MSE | Precision P(%) |
|---|---|---|
| Expression 2 | 3.0801 ± 5.6485 | 85.15 |
| Actical AEE1 | 3.3191 ± 6.0498 | 84.26 |
| Actical AEE2 | 3.9773 ± 6.5110 | 82.07 |

The above table, in which values includes all data determined as abnormal values, shows that the MSE of the expression 2 is smaller than that of the Actical. Therefore, the present exemplary embodiment predicts more precisely than the reference calorie (kcal) of the metabolic gas analyzer (K4B2) and has the highest precision (P) of 85.15%.

59 subjects wore the metabolic gas analyzer (K4B2), the Actical, and the activity monitor corresponding to the apparatus for calculating the calorie consumption using the 3-axial accelerometer according to the present exemplary embodiment and were tested with respect to his/her various walking speeds in accordance with the test protocol, and the activities AEE1 and AEE2 measured in the Actical were compared with the activity obtained by the expression 2 according to the present exemplary embodiment.

In result, it is appreciated that the performance of the apparatus for calculating the calorie consumption using the 3-axial accelerometer is better than that of the Actical with respect to the calorie (Kcal) of the metabolic gas analyzer (K4B2).

As described above, the apparatus and method for calculating the calorie consumption using the 3-axial accelerometer were explained as the apparatus for calculating the calorie consumption using the high band-pass digital filter for performing the high band-pass digital filtering, i.e., the LWDF, and the method using the same.

However, the apparatus and method for calculating the calorie consumption may involve a relatively large amount of calculation. To reduce the amount of calculation, the apparatus for calculating the calorie consumption may be configured without the high band-pass filter, i.e., the LWDF, and the method of calculating the calorie consumption using the apparatus for calculating the calorie consumption without the LWDF has the following characteristics.

The method of calculating the calorie in real time using the 3-axial accelerometer may be performed by the activity monitor that is provided with a 3-axial accelerometer, obtains the sum of energy converted from the output values of the 3-axial accelerometer corresponding to a user's physical activity for a preset time, obtains the energy consumption corresponding the user's physical activity on the basis of the sum of energy and a user's weight.

First, the sum of energy converted from the output values of the 3-axial accelerometer corresponding to a user's physical activity for the preset time is obtained. At this time, the expression 1 may be used to convert each output value of the 3-axial accelerometer corresponding to a user's physical activity into the energy. At this time, Ei is energy calculated from the respectively converted signals of the 3-axial accelerometer, and aₓ_{ᵢ}², a_{y}_{ᵢ}², a_{z}_{ᵢ}² are values obtained by raising the acceleration data of the x-, y- and z-axes to the power. Further, i indicates ith data.

Then, the energy consumption corresponding to a user's physical activity is obtained on the basis of the sum of energy and a user's weight. At this time, the energy consumption corresponding to a user's activity can be obtained by the following expression 6.

E=A ln S−BW [Expression 6]

where, E is the energy consumption corresponding to a user's activity, A and B are real numbers, S is the sum of energy converted from the output values of the 3-axial accelerometer corresponding to a user's physical activity for the preset time, and W is a user's weight. Further, A may be 0.1002 and B may be 1.525.

Meanwhile, the method of calculating the calorie in real time using the 3-axial accelerometer according to the present exemplary embodiment may perform zero adjustment for the 3-axial accelerometer before obtaining the sum of energy converted from the output values of the 3-axial accelerometer.

The energy consumption converting performance of the activity motor to which the method of calculating the calorie in real time using the 3-axial accelerometer according to the present exemplary embodiment is applied, and a process of deriving the expression 6 for obtaining the energy consumption to be applied in the activity monitor will be described.

To obtain experimental data, healthy adjusts were recruited as participants in the experiments, and thus 59 men and women between the ages of 21 and 38 were selected. Such subjects have the weights of 49.70 kg to 115.70 kg and an average age of 28. The characteristics of the subjects participated in the present experiments are as shown in FIG. 8, and the acceleration output data of various walking speeds in a treadmill was obtained and tested.

The subjects wore a metabolic gas analyzer (K4B2) and attached the activity monitor according to the present exemplary embodiment to his/her right upper arm and right waist. Further, the Actical also were attached to the left waist, and then the subjects changed speed in order of easy walking, power walking, light running, running and fast running on the treadmill for 5 minutes per step. The test protocol was acquired through consultation with an exercise physiologist, and interval training of 1 minute was given between the steps in consideration of time to be taken until breathing becomes steady, which were as shown in the following table 2. Taking a physical attribute into account, the treadmill speed of the female was set to be slower by 1 km/h than that of the male.

There is little difference in data output whether the accelerometer is attached to the left or right arm (refer to “N. Twomey, S. Faul, W. P. marnane, Comparison of accelerometer-based energy expenditure estimation algorithms, Pervasive Computing Technologies for Healthcare 4th international conference on, pp 1-8, 2010”). In this exemplary embodiment, the accelerometer was attached to the right waist.

Below, a process of deriving a formula for obtaining a user's energy consumption by using energy converted from the output values of the 3-axial accelerometer corresponding to a user's physical activity based on the test protocol as shown in the table of FIG. 9 will be described.

The 3-axial accelerometer was zero-adjusted using a simple 0g x, 0g y, +1g z calibration method. Because the output values of the 3-axial accelerometer contain a rotation component, the output values was converted into the energy through the foregoing expression 1 so that the rotation component can be processed as one representative value without being considered.

To be matched with the data acquired by the metabolic gas analyzer (K4B2) and the conventional activity monitor, i.e., the Actical, accelerometer raw data was processed in the present activity monitor like the following expression 3. Here, n is 1920 as data for one minute, and S is the sum of energy.

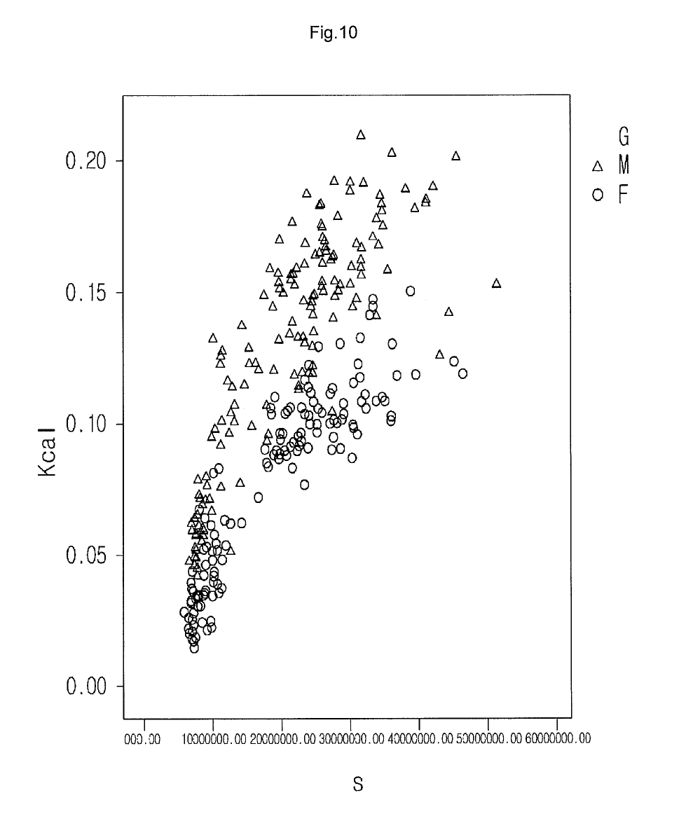

To derive a regression formula, a scatter diagram was drawn using the data acquired through the experiment. FIG. 10 is a scatter diagram showing calorie (Kcal) based on gender and S obtained by the expression 3. Here, “0” indicates the male, and “1” indicates the female. It will be appreciated that the male shows higher calorie (Kcal) than the female with respect to the same S. Considering that the calorie (Kcal) is largely dependent on the weight, it is natural since the female has smaller weight than the male.

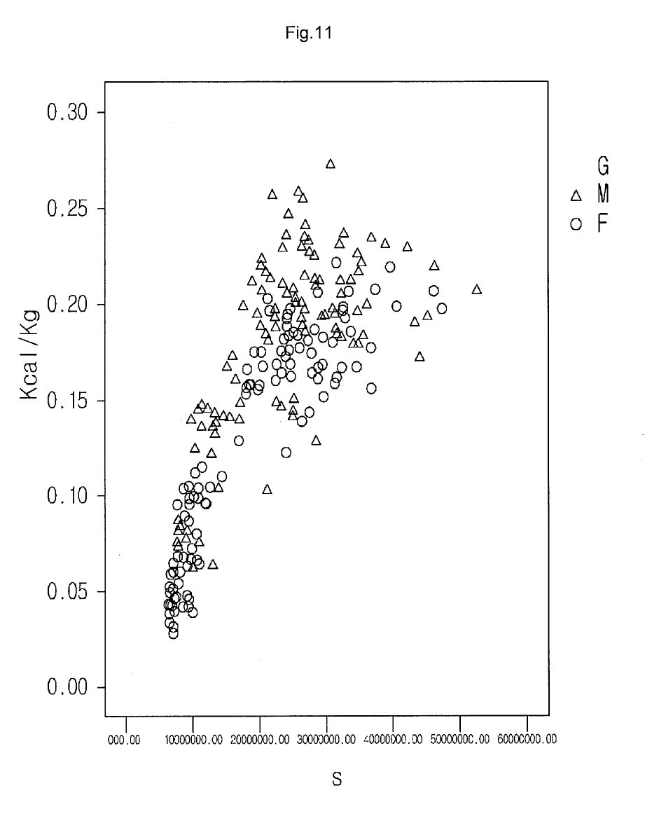

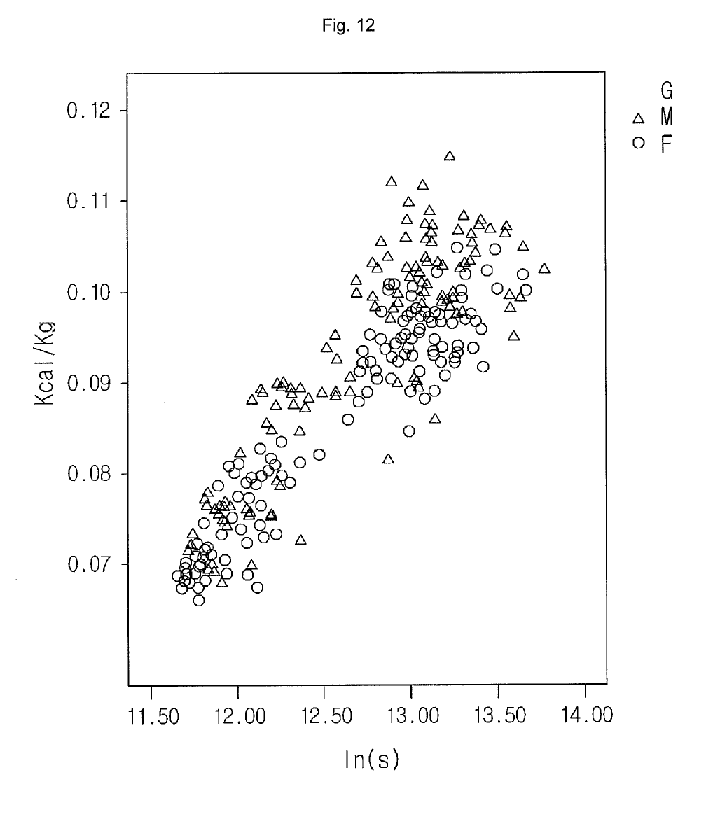

Thus, if a scatter diagram is drawn with S and the calorie (Kcal) divided by the subject's weight, the scatter diagram shows uniform distribution regardless of gender as shown in FIG. 11. However, it will be appreciated that Kcal/Kg and S are not liner. Therefore, they have to undergo variable conversion and change to be linear in order to apply a linear regression analysis thereto. In FIG. 11, the scatter diagram shows a log type. Thus, it will be understood that a linear relationship is shown when ln is applied to S. As shown in the scatter diagram of FIG. 12, there is the linear relationship between Kcal/Kg and ln(s). Actually, a correlation coefficient between two variables is r=0.983, which very approximates to 1, thereby showing the linear relationship.

To perform the linear regression analysis for Kcal/Kg and ln(s) obtained through the variable conversion, a linear regression model of the following expression 7 was applied.

Yᵢ=α+βXᵢ+eᵢ, i=1,2, . . . ,n [Expression 7]

where, α is a regression coefficient, parameter and intercept; β is a regression coefficient, a gradient an explanation variable x, and increase (i.e., a differential coefficient) of a dependent variable y every time when the explanation variable x increases by one step; Y is Kcal/Kg; X is explanation variable (S); and e is an error term.

To estimate the regression model of the expression 7, α and β of the expression 7 minimizing

$${{\sum }_{i = 1}}^{n} {e}_{i}^{2} = 0$$

are estimated using the ordinary least square, the estimation values \hat{α}, \hat{β} R minimizing Q of the expression 8 are partially differentiated to solve the normal equations resulting in 0 such as the expressions 9 and 10, thereby obtaining the expressions 11 and 12.

$$\begin{matrix}Q = {\sum }_{i = 1}^{n} {e}_{i}^{2} = {\sum }_{i = 1}^{n} {( {Y}_{i} - \hat{α} - \hat{β} {X}_{i} )}^{2} & [ Expression 8 ] \\ \frac{\partial Q}{\partial α} = - 2 {\sum }_{i = 1}^{n} {( {Y}_{i} - \hat{α} - \hat{β} {X}_{i} )}^{2} = 0 & [ Expression 9 ] \\ \frac{\partial Q}{\partial α} = - 2 {\sum }_{i = 1}^{n} {{X}_{i} ( {Y}_{i} - \hat{α} - \hat{β} {X}_{i} )}^{2} = 0 & [ Expression 10 ] \\ \hat{α} = \overline{Y} - \hat{β} \overline{X} & [ Expression 11 ] \\ \hat{β} = \frac{{\sum }_{i = 1}^{n} ( {X}_{i} - \overline{X} ) ( {Y}_{i} - \overline{Y} )}{{\sum }_{i = 1}^{n} {( {X}_{i} - \overline{X} )}^{2}} & [ Expression 12 ] \\ {\hat{Y}}_{i} = \hat{α} + \hat{β} {X}_{i} & [ Expression 13 ]\end{matrix}$$

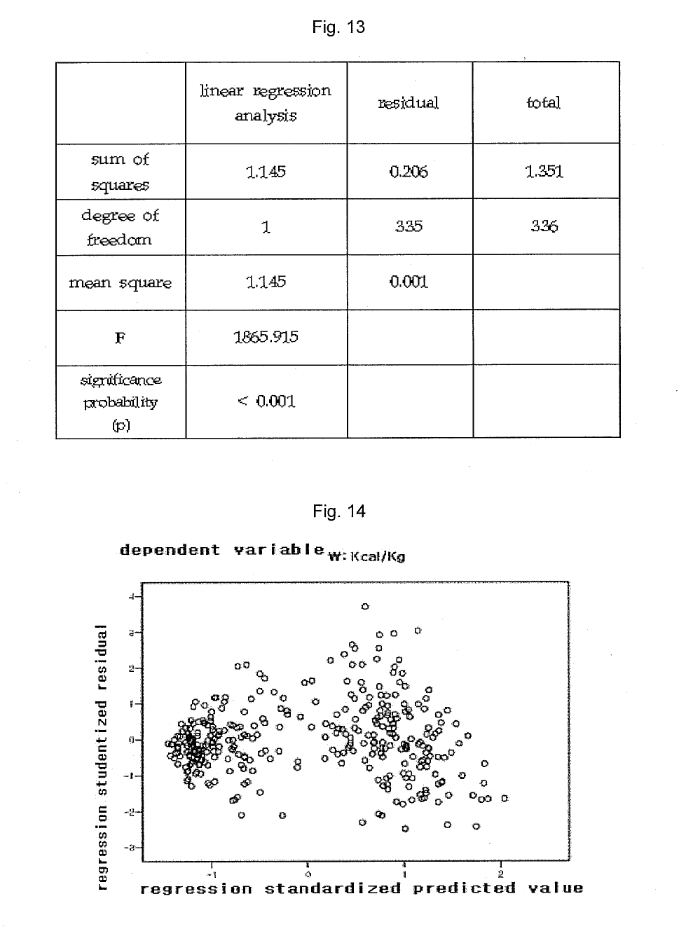

The expression 13 is a regression equation obtained using the least square values of the expressions 11 and 12. To estimate a gradient regression coefficient (β) of the expression 7, a significant test for the explanation variables was performed. To this end, a hypothesis test about a null hypothesis H_{O}=β:O is as shown in the table of FIG. 13. It is understood that the P value (significant probability) is significant since it is smaller than 0.05.

As shown in FIG. 14, there is no special pattern having no linearity and no homoscedasticity. A value having a residual of 2 or higher is eliminated by analyzing a studentized residual, and the filtering is performed ten times, thereby deriving the regression equation as shown in the expression 6. There were 337 measured values, but 101 values were determined as the abnormal values, so that only 236 data were used to implement the regression analysis. The reason why there are many abnormal values may be because everybody has different walking or running patterns. At this time, the derived regression equation, i.e., the expression 6 satisfies t=81.329, p<0.001 and R2=0.966.

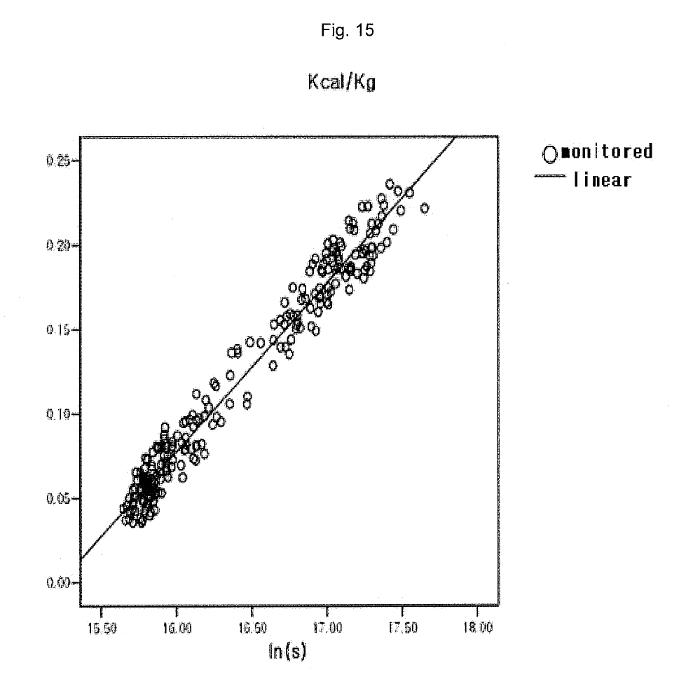

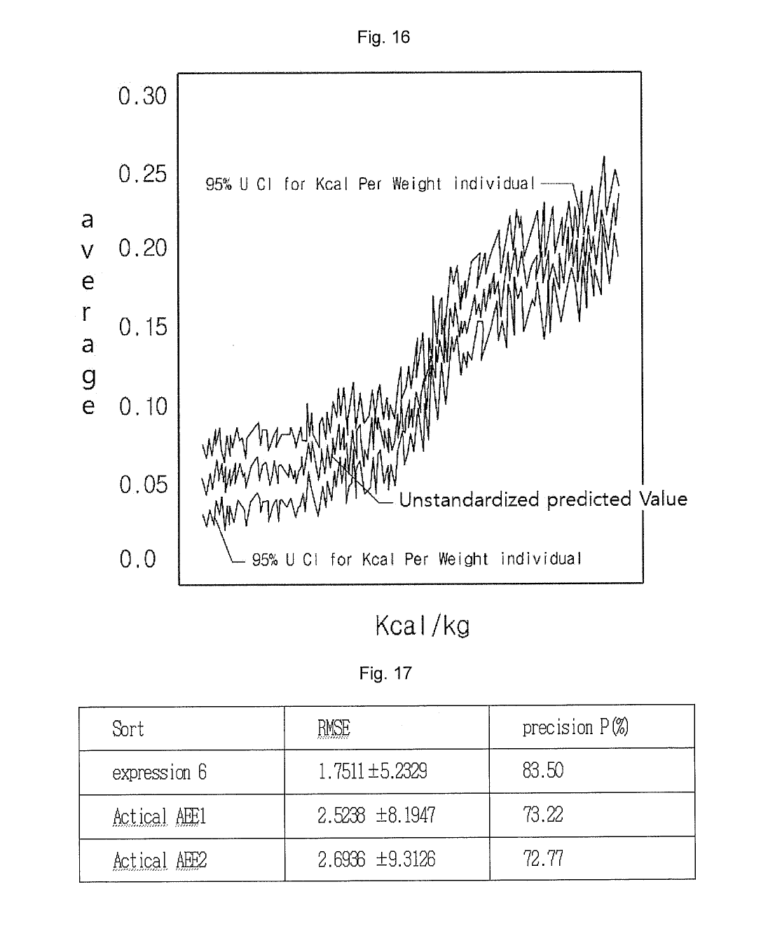

FIG. 15 is a graph showing a linear relationship between the regression equation and the real measured value, and FIG. 16 is a graph showing a 95% confidence interval. The performances of the expression 6 applied to the method for calculating the calorie according to the present exemplary embodiment, and the AEE1 and AEE2 of the conventional activity monitor, the Actical were tabulated in the table shown in FIG. 17 by obtaining the root mean square error (RSME) as shown in the expression 14 and obtaining the precision to the real calorie (Kcal) from the metabolic gas analyzer as shown in FIG. 17.

$$\begin{matrix}RMSE = \frac{1}{n} {\sum }_{i = 1}^{n} {( {Y}_{i} - {\hat{Y}}_{i} )}^{2} & [ Expression 14 ] \\ P = \frac{1}{n} {\sum }_{i = 1}^{n} ( {Y}_{i} - \frac{\vert {Y}_{i} - {\hat{Y}}_{i} \vert}{{Y}_{i}} ) \times 100 & [ Expression 15 ]\end{matrix}$$

n: The number of measured values

Yᵢ: Real calories

Ŷᵢ: Predicted calories

The table of FIG. 17, in which values includes all data determined as abnormal values, shows that the MSE of the expression 6 is smaller than that of the Actical. Therefore, the present exemplary embodiment predicts more precisely than the reference calorie (Kcal) of the metabolic gas analyzer (K4B2) and has improved precision (P) by about 10%.

59 subjects wore the metabolic gas analyzer (K4B2), the Actical, and the activity monitor according to the present exemplary embodiment and were tested with respect to his/her various walking speeds in accordance with the test protocol, and the activities AEE1 and AEE2 measured in the Actical were compared with the activity obtained by the expression 6 according to the present exemplary embodiment. In result, it is appreciated that the performance of the proposed algorithm is better than that of the Actical with respect to the calorie (Kcal) of the metabolic gas analyzer (K4B²).

As described above, there are provided an apparatus and method for calculating calorie consumption using a 3-axial accelerometer, in which an output value of the 3-axial accelerometer and a predetermined mathematical expression are used to calculate a user's calorie consumption, a lattice wave digital filter is further used to calculate energy without being affected by the acceleration of gravity, and the energy consumption (Kcal) corresponding to a user's physical activity can be calculated at a high degree of precision by taking the above energy and a user's weight and gender into account.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. An apparatus for calculating calorie consumption using a 3-axial accelerometer, the apparatus comprising:
the 3-axial accelerometer which outputs acceleration signals corresponding to a user's physical activity with respect to x-, y- and z-axes;
a low band-pass filter which applies low band-pass filtering to the output signals of the accelerometer with respect to each axis;
a high band-pass digital filter which applies high band-pass digital filtering to the output signals of the accelerometer filtered by the low band-pass filter; and
a calorie consumption calculator which calculates a user's calorie consumption on the basis of the output signal of the accelerometer filtered by the high band-pass digital filter.

2. The apparatus according to claim 1, wherein the low band-pass filter comprises a 2^{nd} order analog low pass filter which applies 2^{nd} order analog filtering to the output signal of the accelerometer with respect to each axis, and a digital low pass filter which applies digital filtering to the output signal of the accelerometer with respect to each axis filtered by the 2^{nd} order analog low pass filter.

3. The apparatus according to claim 1, wherein the high band-pass digital filter comprises a lattice wave digital filter (LWDF), and the lattice wave digital filter comprises a 5 Hz high-pass digital filter.

4. The apparatus according to claim 1, wherein the calorie consumption calculator comprises an energy calculator which calculates the sum of energy for a preset time on the basis of the output signal of the accelerometer filtered by the high band-pass digital filter, and an energy consumption calculator which calculates energy consumption corresponding to a user's physical activity on the basis of the sum of energy calculated by the energy calculator and a user's weight and gender.

5. The apparatus according to claim 4, wherein the energy calculator calculates the sum of energy by the following expression:
Eᵢ=\sqrt{αₓ_{ᵢ}²+α_{y}_{ᵢ}²+α_{z}_{ᵢ}²} [Expression]
where, Ei is energy calculated from the respectively converted signals of the 3-axial accelerometer, and aₓ_{ᵢ}², a_{y}_{ᵢ}², a_{z}_{ᵢ}² are values obtained by raising the signals of the 3-axial accelerometer, respectively converted from the accelerometer signals of the x-, y- and z-axes by the lattice wave digital filters, to the power.

6. The apparatus according to claim 4, wherein the energy consumption calculator calculates the energy consumption corresponding to a user's physical activity by the following expression:
Y=W(A+BG)X^{(C-DG)} [Expression]
where, Y is energy consumption corresponding to a user's physical activity; A, B, C and D are real numbers; X is the sum of energy calculated from the signals of the 3-axial accelerometer passed through the lattice wave digital filters corresponding to a user's physical activity for the preset time; and W is a user's weight (kg), and G is a gender index having a value of 0 or 1.

7. The apparatus according to claim 6, wherein A is 4.488, B is 1.869, C is 0.722, and D is 0.095.

8. The apparatus according to claim 6, wherein G is 0 if a user is a male and 1 if a user is a female.

9. A method for calculating calorie consumption using a 3-axial accelerometer, the method comprising:
receiving output signals of the 3-axial accelerometer corresponding to a user's physical activity and outputting 3-axial accelerometer signals converted by a lattice wave digital filter (LWDF);
calculating the sum of energy for a preset time on the basis of the converted 3-axial accelerometer signals; and
calculating energy consumption corresponding to a user's physical activity on the basis of the calculated sum of energy and a user's weight and gender.

10. The method according to claim 9, wherein the lattice wave digital filter comprises a 5 Hz high-pass digital filter.

11. The method according to claim 9, wherein the calculating the sum of energy for a preset time on the basis of the converted 3-axial accelerometer signals comprises calculating the sum of energy by the following expression:
Eᵢ=\sqrt{αₓ_{ᵢ}²+α_{y}_{ᵢ}²+α_{z}_{ᵢ}²} [Expression]
where, Ei is energy calculated from the respectively converted signals of the 3-axial accelerometer, and aₓ_{ᵢ}², a_{y}_{ᵢ}², a_{z}_{ᵢ}² are values obtained by raising the signals of the 3-axial accelerometer, respectively converted from the accelerometer signals of the x-, y- and z-axes by the lattice wave digital filters, to the power.

12. The method according to claim 9, wherein the calculating energy consumption corresponding to a user's physical activity on the basis of the calculated sum of energy and a user's weight and gender comprises calculating the energy consumption corresponding to a user's physical activity by the following expression:
Y=W(A+BG)X^{(C-DG)} [Expression]
where, Y is energy consumption corresponding to a user's physical activity; A, B, C and D are real numbers; X is the sum of energy calculated from the signals of the 3-axial accelerometer passed through the lattice wave digital filters corresponding to a user's physical activity for the preset time; and W is a user's weight (kg), and G is a gender index having a value of 0 or 1.

13. The method according to claim 6, wherein A is 4.488, B is 1.869, C is 0.722, and D is 0.095.

14. The method according to claim 12, wherein G is 0 if a user is a male and 1 if a user is a female.

15. A method for calculating calorie consumption using a 3-axial accelerometer, the method comprising:
calculating the sum of energy converted from output values of the 3-axial accelerometer corresponding to a user's physical activity for a preset time; and
calculating energy consumption corresponding to a user's physical activity on the basis of the calculated sum of energy and a user's weight.

16. The method according to claim 15, wherein the calculating the sum of energy comprises converting each output value of the 3-axial accelerometer corresponding to a user's physical activity into energy by the following expression:
Eᵢ=\sqrt{αₓ_{ᵢ}²+α_{y}_{ᵢ}²+α_{z}_{ᵢ}²} [Expression]
where, Ei is energy converted from each output value of the 3-axial accelerometer corresponding to a user's physical activity, and aₓ_{ᵢ}², a_{y}_{ᵢ}², a_{z}_{ᵢ}² are values obtained by raising the acceleration data of the x-, y- and z-axes to the power.

17. The method according to claim 15, wherein the calculating energy consumption comprises calculating the energy consumption corresponding to a user's physical activity by the following expression:
E=A ln S−BW [Expression]
where, E is the energy consumption corresponding to a user's activity, A and B are real numbers, S is the sum of energy converted from the output values of the 3-axial accelerometer corresponding to a user's physical activity for the preset time, and W is a user's weight.

18. The method according to claim 17, wherein A is 0.1002 and B is 1.525.
